SEQUENCE LISTING (1 sequence)

SEQ ID NO: 1 - protein, 13 aa, Homo sapiens
FFYTPKTRREAED

(19) United States
(12) Patent Application Publication
(10) Pub. No.: US 2005/0025758 A1
(43) Pub. Date: Feb. 3, 2005

(54) Method and compositions for treatment of insulin-dependant diabetes mellitus

(76) Inventors: Leonard Charles Harrison, St. Kilda West (AU); Majella Dempsey-Collier, Spring Gully (AU)

Correspondence Address: SCULLY SCOTT MURPHY & PRESSER, PC, 400 GARDEN CITY PLAZA, GARDEN CITY, NY, 11530, US

(21) Appl. No.: 10/867,149

(22) Filed: Jun. 14, 2004

Related U.S. Application Data
(63) Continuation of application No. 09/269,482, filed on Jun. 7, 1999, now abandoned.

(30) Foreign Application Priority Data
Oct. 1, 1996 (AU) P0 2699

(51) Int. Cl. A61K 38/28

(52) U.S. Cl. 424/94.5; 514/3

(57) ABSTRACT

The present invention provides methods of prophylaxis and treatment of autoimmune disease conditions and agents useful for the same. Particularly, the present invention provides mucosa-mediated tolerance to protect against or ameliorate the symptoms associated with autoimmune pathology. More particularly, the present invention provides a method for preventing clinical insulin-dependent diabetes mellitus (IDDM) or preventing or reducing or ameliorating the effects of cynical IDDM by the aerosol administration of IDDM-associated autoantigens to mucosal surfaces.

The present invention relates generally to a method of prophylaxis and treatment of autoimmune disease conditions and agents useful for same. More particularly, the present invention contemplates mucosa-mediated tolerance to protect against or ameliorate the symptoms associated with autoimmune pathology. Even more particularly, the present invention provides a method for preventing clinical insulin-dependent diabetes mellitus (IDDM) or preventing or reducing or ameliorating the effects of clinical IDDM by the aerosol administration of IDDM-associated autoantigens to mucosal surfaces.

Bibliographic details of the publications numerically referred to in this specification are collected at the end of the description.

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

The increasing knowledge of the immune system in general and cellular immune mechanisms in particular is greatly facilitating the design of therapeutic agents and alternative routes of their administration. One important area of research is the mechanisms underlying cellular immune hyporesponsiveness induced by particular antigens in autoimmune disease conditions.

An autoantigen can be assumed to be pathogenic if its administration modifies the natural history of autoimmune disease. Autoantigen-specific strategies of immune tolerance induction have been shown to favourably modify the natural history of experimental autoimmune disease in rodents (8, 24-28). The presentation of soluble protein antigen to mucosal surfaces, classically via the oral route, results in selective suppression of antigen-specific T cell-mediated, delayed-type hypersenisitivity (DTH) and IgE responses (2, 8, 29). ‘Oral tolerance’ has been associated with deviation of immunity away from T-cell (Th1) to antibody (Th2) responses, with the induction of regulatory T cells and, at higher antigen doses, with both T-cell anergy and T-cell deletion (8, 30).

A particularly debilitating autoimmune condition is insulin-dependent diabetes mellitus (IDDM) which results from the selective destruction of insulin-producing β cells in the islets of the pancreas, within an autoimmune inflammatory ‘insulitis’ lesion (10, 11). The primary role or autoreactive T cells in mediating β-cell destruction has been shown directly in two spontaneous animal models of IDDM, the Bio-Breeding (BB) (31) rat and the non-obese diabetic (NOD) mouse (24). Target autoantigens that trigger or drive immune reactivity to β cells not only have diagnostic applications but are potential agents for specific immunotherapy (25-28). Several potentially pathogenic islet/β-cell autoantigens have been identified by their reactivity with circulating antibodies or T cells in rodents and humans with sub-clinical or clinical IDDM, in. particular insulin, glutamic acid decarboxylase (GAD) and tyrosine phosphatases of the IA-2 family (32). However, insulin and its precursor, pre-proinsulin, are the only IDDM autoantigens that are β-cell specific.

Insulin autoantibodies (IAA) are a risk marker for the development of clinical IDDM (33) and have been detected before autoantibodies to other islet antigens in the offspring of diabetic mothers (34). Increased proliferation of peripheral blood T cells to human insulin can be demonstrated in up to half of sub-clinical and recently-diagnosed IDDM subjects (35), but responses are relatively low. This is possibly because the dominant human T-cell epitope is in proinsulin. A peptide that spans the natural cleavage site between the B chain of insulin and the connecting (C) peptide in proinsulin was reported to elicit T-cell proliferation in a majority of at-risk IDDM relatives (36). In the NOD mouse, IAA are reported to be a risk marker for the development of diabetes (37) and the majority of T-cell clones generated from the insulitis lesion reacted to insulin B-chain, amino acids 9-23 (38).

Despite widespread interest in the possibility of preventing IDDM, relatively few studies have evaluated mucosa-mediated tolerance in the natural NOD mouse model. Zhang et al. (22) found that oral porcine insulin (1 mg twice weekly) delayed the onset and reduced the incidence of diabetes, and was associated with splenic T cells that partially blocked the transfer of diabetes to young, non-diabetic mice by spleen cells from diabetic mice. Subsequently, Bergerot et al. (23) reported that the regulatory cells induced by oral insulin were CD4+ T cells. However, in studies of oral tolerance to guinea pig myelin basic protein (MBP) in the Lewis rat model of experimental autoimmune encephalomyclitis (EAE) (8), both CD4 and CD8 regulatory T cells which secrete IL-4, IL-10 and TGF-β have been described.

There is a need to develop effective administration strategies for delivery of antigens to induce suppression of cell-medicated autoimmune conditions. The administration strategies must not only to be immunologically effective but also convenient, direct and safe for the user. In work leading up to the present invention, the inventors investigated the aerosol administration of insulin and its precursors in an animal model for IDDM and showed that this was effective in reducing pancreatic islet pathology and incidence of diabetes.

Accordingly, one aspect of the present invention contemplates a method of suppressing a cell-mediated autoimmune disease in a subject, said method comprising the administration as an aerosol of an effective amount of an antigen associated with said autoimmune disease for a time and under conditions sufficient to prevent, reduce or otherwise ameliorate autoimmune pathology.

More particularly, the present invention provides a method of preventing, reducing or otherwise ameliorating an autoimmune disease condition in a subject, said method comprising the administration, as an aerosol, to said subject of an effective amount of an antigen associated with said autoimmune disease for a time and under conditions sufficient to induce or stimulate immunoregulatory mechanisms which are protective against cell-mediated autoimmune pathology.

Reference hereinafter to “immunoregulatory mechanisms” should be understood as a reference to all mechanisms which regulate cell-mediated immune responses including, but not limited to, regulation of T cell functional activity, for example regulation of one or more of suppressor T cells, Th1, Th2 or CD8⁺ T cells (referred to herein as “regulatory T cells”), or via regulation of cytokine production by lymphoid, myeloid or stromal cells.

The present invention is hereinafter described with respect to preventing, reducing or otherwise ameliorating IDDM, slowly progressive IDDM (SPIDDM) also referred to as latent autoimmune diabetes in adults [LADA] and gestational diabetes due to underlying IDDM. This is done, however, with the understanding that the present invention extents to a range of cell-mediated autoimmune conditions.

Accordingly, another aspect of the present invention contemplates a method of preventing, reducing or otherwise ameliorating IDDM, SPIDDM or gestational diabetes in a subject, said method comprising the administration, as an aerosol, to said subject of an effective amount of an autoantigen associated with IDDM for a time and under conditions sufficient for induction of regulatory T-cells and/or other suitable mechanisms sufficient to suppress cell-mediated autoimmune pathology associated with IDDM.

Reference hereinafter to “IDDM” includes IDDM, SPIDDM and gestational IDDM.

The regulatory T cells induced will depend on the form of antigen and its route of administration. For example, when a long peptide or whole molecule is administered (eg. insulin), CD8 T cells and, more particularly, CD8γδ T cells are induced. Smaller molecules such as proinsulin peptides (eg. proinsulin peptide 24-36) generally induce CD4 T cells and, more particularly, CD4αβ T cells.

A particularly preferred form of administration is intranasal administration via an aerosol spray, drip or vapour.

The preferred antigen associated with IDDM used for aerosol or other intranasal administration composition is preproinsulin or proinsulin as well as insulin and their immune response stimulatory derivatives thereof such as but not limited to peptide fragments of proinsulin, (eg. proinsulin peptide 24-36), preproinsulin or insulin. Immune response stimulation preferably includes regulatory T cell stimulation. However, any islet antigen may be employed such as, but not limited to, glutamic acid decarboxylase (GAD) in its various isoforms (for example GAD 65 and GAD 67) or derivatives thereof and tyrosine phosphatase IA-2 or derivatives thereof. Said antigens may be from human or any non-human species such as mouse.

The term “derivatives” includes fragments, parts, portions, chemical equivalents, mutants, homologs and analogs of the antigens. Analogs may be derived from natural synethic or recombinant sources and include fusion proteins. Chemical equivalents of an antigen can act as a functional analog of an antigen. Chemical equivalents may not necessarily be derived from an antigen but may share certain conformational similarities. Alternatively chemical equivalents may he specifically designed to mimic certain physiochemical properties of an antigen. Chemical equivalents may be chemically synthesised or may be detected following, for example, natural product screenings.

A homolog of an antigen contemplated herein includes but is not necessarily limited to antigens derived from human or any non-human species such as mouse.

Derivatives include one or more insertions, deletions or substitutions of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in said cytokines although random insertion is also possible with suitable screening of the resulting product. Deletional variants are characterised by the removal of one or more amino acids from the sequence. Substitional amino acid variants are those in which at least one residue in the sequence has been removed and a different residue inserted in its place. Additions to amino acid sequences include fusions with other peptides or polypeptides. It is possible, for example, that the subject preferred cytokines may be substituted by other cytokines or lymphoid cytokines or functional homologs or analogs. A hybrid cytokine may comprise a combination of cytokines.

Said derivatives include peptide derivatives and peptide epitope analogs. Reference to a “disease suppressing peptide comprising proinsulin 24-36 sequence” should be understood to include reference to a proinsulin 24-36 sequence to which flanking amino acid sequences have been added to one or both ends of said proinsulin 24-36 sequence. Said flanking ends may be useful, for example, for enhancing the effectiveness of the proinsulin 24-36 sequence by a mechanism such as increasing the hydrogen bonding of said proinsulin 24-26 sequence to MHC molecules. Said flanking ends may comprise, for example, alanine residues which are added to the C terminus of said proinsulin 24-36 sequence.

The term “aerosol” is used in its most general sense to include any formulation capable of administration via nasal, pharyngeal, bronchial or oral passages. Aerosols generally comprise particles of liquid or solid suspended in a gas or vapour. Conveniently, the aerosol is a colloidal system such as a mist in which the dispersion medium is a gas. The method of administering the aerosol formulation is not critical and may be achieved using a hand pump, electric pump, pressurised dispenser, nasal drip or other convenient means. It should be understood that the method of the present invention extends to direct application of said formulations to intranasal surfaces. In a particularly preferred embodiment, the aerosol is delivered at a rate of from about 1 to about 20 litres/min. and preferably from about 2 to about 15 litres/min. at a droplet size of from about 0.1 to about 10 μm and more preferably from about 0.1 to about 6 μm. Conveniently, a stock solution of antigen is prepared at a concentration of from about 0.5 to about 20 mg/ml or more preferably from about 1.0 to about 10 mg/ml of carrier solution. Commercially available insulin is particularly useful which is about 4 mg/ml. A useful dose is from about 50 μl to 1000 μl and preferably 100 μl to 500 μl from the stock solution.

The antigen may he administered alone or by formulation in or with an adjuvant. The adjuvant is selected from a range of adjuvants which enhance an immunoregulatory response including cholera toxin B, heat labile toxin of E. coli, saponin, Quill A extracts and other derivatives of saponin, DEAE-dextran, dextran sulphate, aluminium salts, and nonionic block co-polymers. The adjuvant may include other immunomodulators, such as cytokines (for example IL-4 or IL-13), muramyl-dipeptide and derivatives, and cell wall components from species of mycobacteria or corynebacteria. The adjuvant formulation may include a combination of two or more of the adjuvants listed. These lists are not to be taken as exhaustive. The selection of adjuvant is in part dependent on the species being targeted and is based on the level and duration of the immune response required and oil the lack of reactogenicity (ie tissue compatibility). The level of active component and adjuvant are chosen to achieve the desired level and duration of immune response.

The antigen is administered in a therapeutically effective amount. A therapeutically effective amount means that amount necessary at least partly to attain the desired effect, or to delay the onset of, inhibit the progression of, or halt altogether, the onset or progression of the particular condition being treated. Such amounts will depend, of course, on the particular conditions being treated, the severity of the condition and individual patient parameters including age, physical conditions, size, weight and concurrent treatment. These factors are well known to those of ordinary skill in the art and can be addressed with no more than routine experimentation. It is preferred generally that a maximum dose he used, that is, the highest sale dose according to sound medical judgement. It will be understood by those of ordinary skill in the art, however, that a lower dose or tolerable dose may be administered for medical reasons, psychological reasons or for virtually any other reasons.

Generally, daily oral doses of antigen will be from about 0.01 mg/kg per day to 1000 mg/kg per day. Small doses (0.01-1 mg) may be administered initially, followed by increasing doses up to about 1000 mg/kg per day. In the event that the response in a subject is insufficient at such doses, even higher doses (or effective higher doses by a different, more localised delivery route) may be employed to the extent patient tolerance permits. A single dose may be administered or multiple doses may be required on an hourly, daily, weekly or monthly basis. Effective amounts of antigen vary depending on the individual but may range from about 0.1 μg to about 20 mg, preferably from about 1 μg to about 10 mg and more preferably from about 1 μg to 5 mg per dose.

In a related aspect of the present invention the subject undergoing treatment may be any human or animal in need of therapeutic or prophylactic treatment.

The immune status generally and specifically levels of regulatory T cells and cytokine profiles, may be readily determined throughout any treatment regime using conventional methods known to those skilled in the art. For example, regulatory T cell levels may be monitored by cytometric analysis following labelling with commercially available antibodies specific to T cell subsets. Other examples of methods suitable for determining the status of the subject include purification of peripheral blood mononuclear cells by density centrifugation followed by stimulation by incubation with well known antigens such GAD, IA-2 family members, insulin or proinsulin. Resulting proliferation may be quantified by assaying for incorporation of H³ thymidine. The cytokine profile can be determined approximately 24 hours after stimulation by antigen. Said cytokines can be detected using, for example, specific cytokine antibodies. 24 hours after stimulation with antigen, stimulated cells can be phenotypically characterised by, for example, flow cytometric analysis of activation marker expression (for example CD69, CD44, CTLA4, CD25). Following cell surface labelling of activated cells, said cells may be further fixed and incubated with fluorochrome labelled antibodies to specific cytokines to determine intracellular cytokine levels. In particular, for example, cells may be further assessed by double labelling assays. The double labelled cells may be analysed utilising flow cytometric analysis or fluorescence spectroscopy.

Another aspect of the present invention provides a composition comprising an antigen associated with an autoimmune disease in an aerosol formulation including one or more pharmaceutically acceptable carriers and/or diluents.

Preferably, the autoimmune disease is IDDM.

Preferably, the antigen is an islet antigen such as insulin, or a precursor thereof such as preproinsulin, proinsulin or their derivatives (eg. proinsulin peptide 24-36) or GAD or tyrosine phosphatases IA-2 or derivatives thereof.

Preferably, the antigen and route of administration induce regulatory T cells, such as in relation to whole molecules such is insulin CD8 T cells and most preferably CD8 γδ T cells or, in relation to smaller molecules such; as proinsulin peptide 24-36, CD4 T cells and most preferably CD4αβ T cells.

The present invention is further described by the following non-limiting Figures and/or Examples.

### IN THE FIGURES

FIG. 4 is a graphical representation showing that aerosol insulin induces CD8 T cells that suppress transfer of diabetes. NOD male mice (n=16/group) aged 6-9 weeks were injected with pooled splenocytes from recently-diabetic 14-19 week old females, together with either unfractionated (A) or fractionated (B-E) splenocytes from aerosol insulin- or ovalbumin-treated NOD females, and their incidence of diabetes subsequently monitored. In the experiment shown, aerosol donor mice had been treated for 10 consecutive days and then weekly from 49 days of age and were normoglycemic when sacrificed at 156 days of age.

FIG. 5 is a graphical representation showing that aerosol insulin induces CD8 γδ T cells that suppress transfer of diabetes. Young male NOD mice were co-injected with ‘diabetic’ splenocytes (2×10⁷) and total or fractionated splenic T cells from aerosol-treated mice, as in the legend to FIG. 1. The numbers of fractionated cells injected were, in A) ˜10⁷ total T cells and, from aerosol insulin mice, ˜10⁷ γδ-depleted T cells or 1.4×10⁵ γδ T cells and, in B), from aerosol insulin mice, ˜10⁷ total T cells, 2×10⁶ CD8 T cells, 2×10⁶ γδ-depleted CD8 T cells or 1.5×10⁵ CD8 γδ+ve T cells.

FIG. 6. is a diagrammatical representation showing that adoptive transfer of diabetes is suppressed by CD8 γδ T cells induced by aerosol insulin: summary of 11 experiments.

### EXAMPLE 1

### Aerosol Treatment and Diabetes Assessment

Semi-sealed boxes of eight female NOD mice were each aerosolized by connection to a standard, patient electric pump (Maymed Aerosol MKV, Anaesthetic Supplies, Sydney, Australia) and Aeroflo nebulizer (Waite & Co., Sydney). Recombinant human insulin (Humulin R, Eli Lilly) or control ovalbumin protein, at 4 mg/ml, was delivered over 10 min. at an air flow rate of 6 litres/min. in a rated droplet size of <5.8 μm, to groups of 24-32 mice. All treatments were given between 0900 and 1100 hours. Protocols and mouse care were approved and supervised by the institutional Animal Ethic Committee. Retro-orbital venous blood was sampled at least every 28 days from 100 days of age and mice considered to be diabetic if their blood glucose, confirmed by a repeat test, was >11 mM. Glucose was measured with BM-Test Glycemic® strips and a Reflolux® II meter (Boehringer-Mannheim), on a drop of blood aspirated via a glass capillary tube from the retro-orbital venous plexus of unanesthetised mice.

### EXAMPLE 2

### Histology

Mice were killed by CO₂ inhalation and the pancreas and salivary glands immediately removed into Bouin's fixative and embedded in paraffin. The insulitis score, a measure of the severity of islet infiltration was determined blindly by two independent investigators by grading and then averaging a minimum of 15 separate islets in serial 6 μm pancreas sections stained with haematoxylin and eosin. The grading scale was: 0, no filtration, islet intact; 1, <10 peri-islet lymphoid cells, islet intact; 2, 10-20 peri-and intra-islet lymphoid cells, islet intact; 3, >20 peri- and intra-islet lymphoid cells, <50% of islet replaced or destroyed; 4, massive lymphoid infiltrate with >50% of islet replaced or destroyed. Infiltration of the salivary glands was graded by the number of lymphoid cells in clusters: 0, no cells; 1, <10 cells; 2, 10-50 cells; 3, >50 cells.

### EXAMPLE 3

### Immune Responses

Spleen cells from individual normoglycemic mice were treated with a red cell lysis buffer, resuspended and incubated in quadruplicate at 2×10⁵/200 μl of serum-free HL-1 medium (Hycor, Irvine, Calif.) containing 50 μm 2-mercaptoethanol, in round-bottom wells with the indicated concentrations of antigen. After 3 days at 37° C. in 5% v/v CO₂/air, 100 μl aliquots from each replicate supernatant were collected and stored at −70° C. for cytokine assays; the cells were then pulsed with 3H-thymidine, harvested 16 hours later and counted on a Topcount™ micro-scintillation counter (Packard, Meriden, Conn.). Insulin was recombinant human (Humulin R, Eli Lilly), is used for aerosol treatments. Insulin B-chain peptide corresponding to amino acids 9-23 of mouse insulin II (Peptide Express, Fort Collins, Colo.) was more than 90% pure by HPLC analysis. GAD65 was the recombinant human form expressed with a C-terminal hexahistidine in a baculovirus system and purified by Ni²⁺ chelation) affinity chromatography. It was resolved as a single band in SDS-PAGE and was endotoxin-free by the quantitative Limulus lysate assay (BioWhittaker, Walkersville, Md.).

IL-2, -4, -10 and IFN-γ were measured by ELISAs with monoclonal antibody pairs (Pharmingen); the lower limits of detection were 62, 16, 16 and 55 pg/ml, respectively. TGF-β1 was measured with an ELISA kit (Promega) with a lower limit of detection of 16 pg/ml.

To detect insulin antibodies, ¹²⁵lablelled human insulin (approx. 100,000 cpm: specific activity 120 μCi/μg) was incubated with or without excess unlabelled insulin (10 μg/ml) in phosphate-buffered saline containing a mixture of protease inhibitors and serial log dilutions of mouse serum, for 5 days at 4° C. Complexes were then precipitated with rabbit anti-mouse globulin anti-serum, washed and counted in a gamma counter. Positive control sera (guinea pig anti-porcine insulin serum, human IDDM sera) maximally precipitated 37-54% of the total radioactivity. Non-specific binding, in the presence of excess unlabelled insulin, was ≦3.3%.

### EXAMPLE 4

### Adoptive Transfer or Diabetes

Male NOD mice aged 6-9 weeks (16/group) were irradiated (800R) from a Cobalt source and 3-6 hours later received 2×10⁷ pooled splenocytes from recently-diabetic 14-19 week-old female NOD mice, together with 2×10⁷ splenocytes (or cells fractionated from this number) from either aerosol insulin- or ovalbumin-treated mice, in 200 μl via the tail vein. The onset of diabetes was then monitored hy measuring blood glucose starting two weeks alter transfer.

### EXAMPLE 5

### Fractionation or Spleen Cell Populations

Spleen cells were treated with red cell lysis buffer and resuspended in mouse tonicity phosphate buffered saline. Total T cells were purified by non-adherence to nylon wool. CD4 and CD8 cells were positively selected/depleted magnetically with monoclonal antibodies directly bound to MACS MicroBeads (Milteny Biotec, GmbH, F.R.G.) according to the manufacturer's protocols, and counted as viable cells (trypan blue stain negative). Flow cytometry revealed 95% depletion o CD4 or CD8 cells, with recoveries ˜80% and ˜50%; respectively.

γδ T cells were positively selected/depleted by incubating T cells from aerosol-treated mice first with biotinylated GL3-1A antibody (Pharmingen, San Diego, Calif.) and then with streptavidin-MACS MicroBeads, followed by magnetic separation. By flow cytometry, γδ cells comprised 1-2% of NOD splenocytes and were totally depleted with GL3-1A antibody. To purify CD8 γδ T cells, CD8 T cells were first magnetically selected from total T cells with anti-CD8-FITC conjugate and anti-FITC MicroBeads. The MicroBeads were then released according to the Miltenyi Biole protocol, and the CD8 cells magnetically separated into γδ positive and depleted fractions. Double staining and FACS analysis demonstrated total depletion of γδ cells and their recovery as a GL3-1A high and low expressing CD8 population.

### EXAMPLE 6

### Diabetes and Instilitis

Aerosol human insulin or ovalbumin were administered in different schedules to female NOD mice from 28 days of age, the earliest time at which insulitis is detectable in the colony of mice, and their incidence of diabetes and severity of insulitis subsequently measured.

The incidence of diabetes was only marginally affected by a single aerosol insulin treatment at 28 days of age, being 75% by 240 days of age compared to 88% after aerosol ovalbumin. However, treatment for 3 or 10 consecutive days and then weekly significantly delayed the onset and reduced the incidence of diabetes. In five separate experiments, diabetes incidence at 156 days of age was reduced from a median of 47% in ovalbumin-treated mice to 23%. in insulin-treated mice; at 240 days of age, when the cumulative incidence of diabetes approaches a maximum, the values were 79% and 49%, respectively (p=0.005, Kaplan-Meier survival statistic). There was no difference if the initial treatment was for 3 or 10 days. In another experiment, in which treatment was given for 10 consecutive days and then weekly, but not started until 49 days of age when insulitis was well-established, aerosol insulin still significantly reduced diabetes incidence at 156 days from 58% to 25% ( (p=0.000). Insulin treatment was associated with a significant reduction in the severity of the islet lesion, as judged by the ‘insulitis score’, which parallelled the decrease in diabetes incidence (Table 1). Infiltration of the salivary glands by lymphoid cells (sialitis), which also occurs in NOD mice, was unaffected by aerosol insulin.

In the absence of absorption-promoting agents, systemic uptake of insulin from the naso-pharyngeal mucosa in humans is insignificant (1). In NOD mice, blood glucose was not altered in the short-term by aerosol insulin. Insulin solutions labellled with 10% Evan's Blue dye were observed to be deposited in the naso-pharynx, trachea and main bronchial divisions, as well as the oesophagus. While it may be difficult, if not impossible, to avoid some gastrointestinal exposure after aerosol or intranasal delivery of soluble protein, delivery into the naso-pharynx alone is sufficient to induce tolerance (2, 3, 4).

### EXAMPLE 7

### Immune Responses

The inventors investigated if (aerosol insulin treatment had altered immune responses to insulin. Unprimed T-cell proliferative responses to islet antigens, including insulin, have been reported in NOD mice (5) but have not always been reproducible (6). Proliferative responses of spleen cells (0.5-2.5×10⁶ ml) from either insulin or ovalbumin-treated mice. aged 56-105 days, to human insulin or ovalbumin (0.2, 2.0, 20 and 40 μg/ml), in different serum-supplemented or scrum-free media varied by less thin two-fold above basal and were usually depressed below basal alt the highest concentration of insulin. Insulin at high concentrations has been reported to inhibit T-cell responses (7). In contrast, in the ovalbumin-treated control mice but not the insulin-treated mice, responses to insulin B-chain peptide a 9-23, a dominant epitope for NOD mouse islet-derived T-cell clones (38), were significant (Table 2). Furthermore, ovalbumin mice had significantly higher responses than insulin mice to human glutamic acid decarboxylase 65 (GAD65), previously reported to stimulate splenic T cells in NOD mice (5). In mice from both treatment groups, proliferative responses to non-antigen-specific stimulation by concanavalin A or T-cell receptor CD3 monoclonal antibody, 145-2C11, were similar (Table 2) and no different to untreated mice, indicating that aerosol treatment did not cause general immunosuppression. IL-2, IFN-γ and TGF-Pδ1 secretion in response to insulin B chain 9-23 were not significantly different between insulin- and ovalbumin-treated mice; however, the levels of IL-4 and particularly IL-10 were higher from cells of insulin-treated mice (Table 3).

Insulin antibodies were measured by a standard immunoprecipitation assay with sera (n=12/group) from insulin- and ovalbumin-treated mice aged 70-105 days. Precipitation of ¹²⁵I-insulin radioactivity by antibodies in sera from insulin-treated mice (12.7±3.6% ; mean precipitated cpm±SD) was significantly higher (p<0.1, Mann Whitney U test) than in ovalbumin-treated mice (6.9±2.5%. This increase in the ‘level’ of insulin antibodies after aerosol insulin, together with the suppression of T-cell proliferation and the increase in IL-4 and IL-10 responses to insulin B-chain peptide, is consistent with the phenomenon of immune deviation, as described after oral MBP in Lewis rats (8) and intranasal GAD peptides in NOD mice (9). β-cell destruction within the DTH lesion of IDDM is an example of Th1-mediated process (10, 11), whose inhibition by aerosol insulin might be expected to shift the Th1/Th2 balance towards Th2 in response to key islet antigens. Defective suppressor T-cell function has been postulated to shift the balance towards Th1 in IDDM (11). It seems unlikely that the reduced T-cell proliferative response to GAD could reflect ‘bystander’ suppression due to the secretion of the Th2 cytokines IL4 and IL-10 (8) by insulin aerosol-induced regulatory cells because, apart from an absence of added insulin in the cultures with GAD, responses to conA and anti-CD3 were not impaired. A direct explanation is that tile reduced response to GAD reflects the protective effect of aerosol insulin on insulitis and β-cell destruction. This implies that at least some GAD immunity is secondary and that immunity to (pro)insulin may have a more proximal role in β-cell destruction. Although NOD mouse T-cell responses to human GAD65 have been reported to be stronger and to appear earlier than those to native human insulin (5), it was found that transgenic expression of mouse proinsulin II in NOD mouse antigen presenting cells completely prevents insulitis and diabetes (12).

### EXAMPLE 8

### Regulatory CD8 γδ T Cells

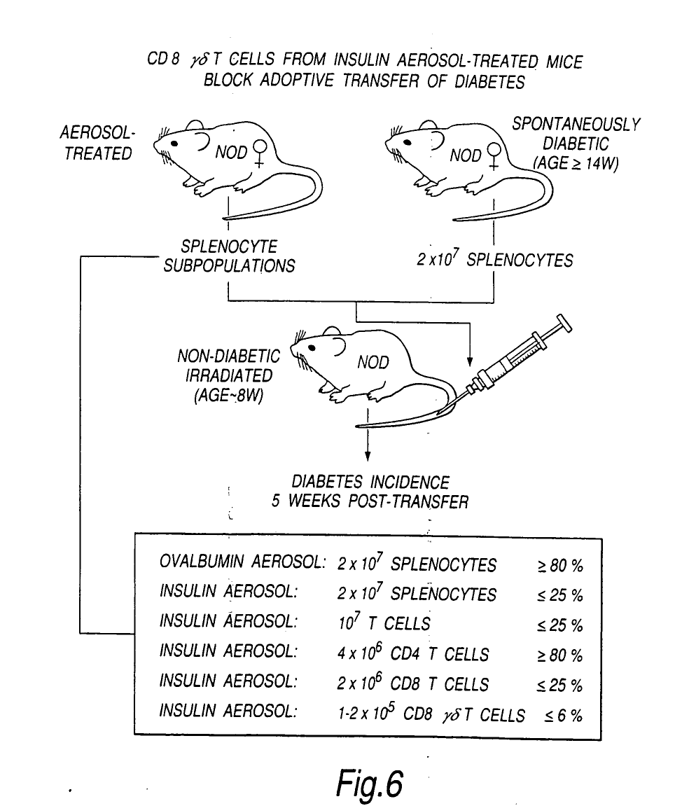

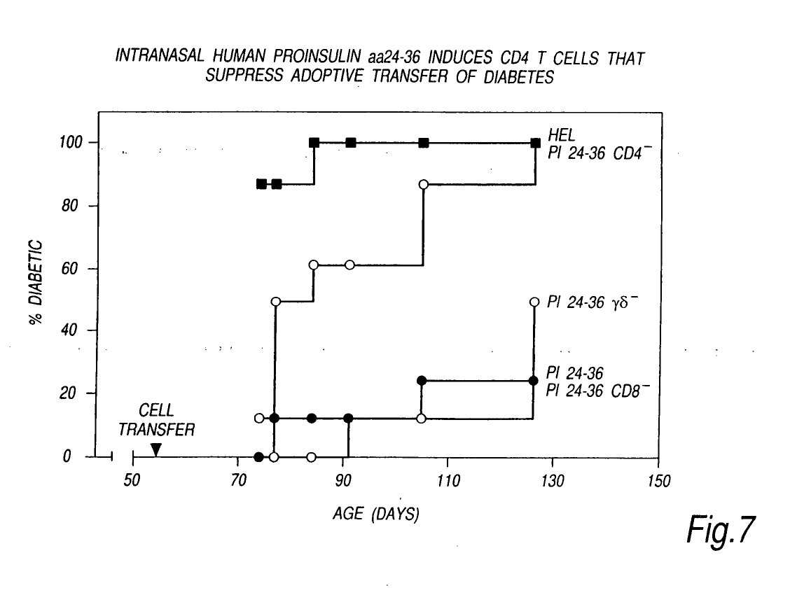

The inventors investigated whether aerosol insulin induced regulatory cells that could inhibit the adoptive transfer of diabetes by pathogenic, effector T cells. In the classic adoptive transfer model (13) (see FIG. 6), spleen cells from diabetic NOD female mice transferred intravenously to young, irradiated non-diabetic syngeneic male or female recipients cause clinical diabetes in tile majority within 4 weeks. When 2×10⁷ spleen cells were co-injected from older, diabetic mice with an equal number of spleen cells from aerosol ovalbumin mice, the majority or young recipients developed diabetes within 4-5 weeks; in contrast, after co-injection with spleen cells from aerosol insulin mice, only a minority developed diabetes (FIG. 1A). Diabetes incidence was suppressed by ≧75% in six separate experiments with either splenocytes or nylon wool-non-adherent splenocytes (enriched for T cells) from aerosol insulin mice.

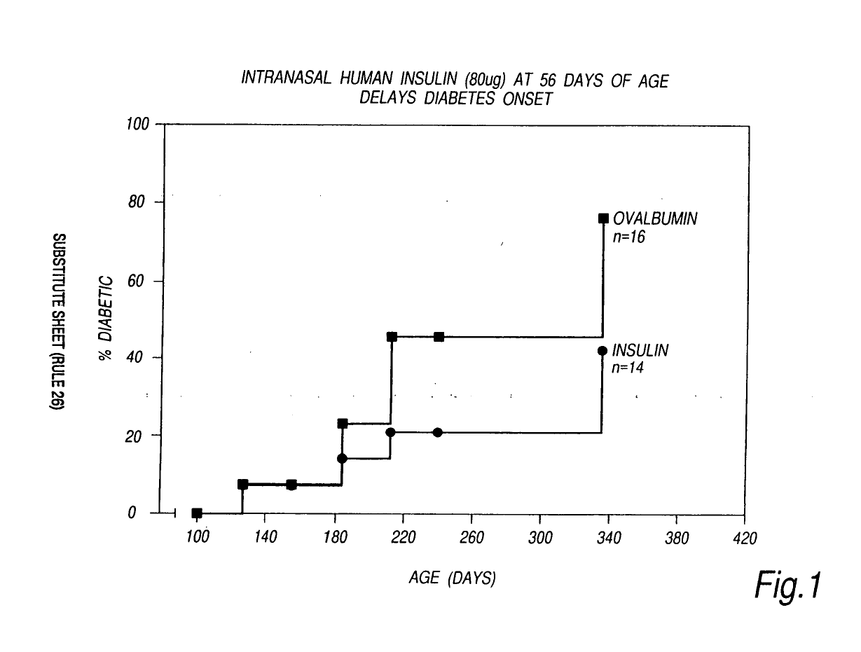

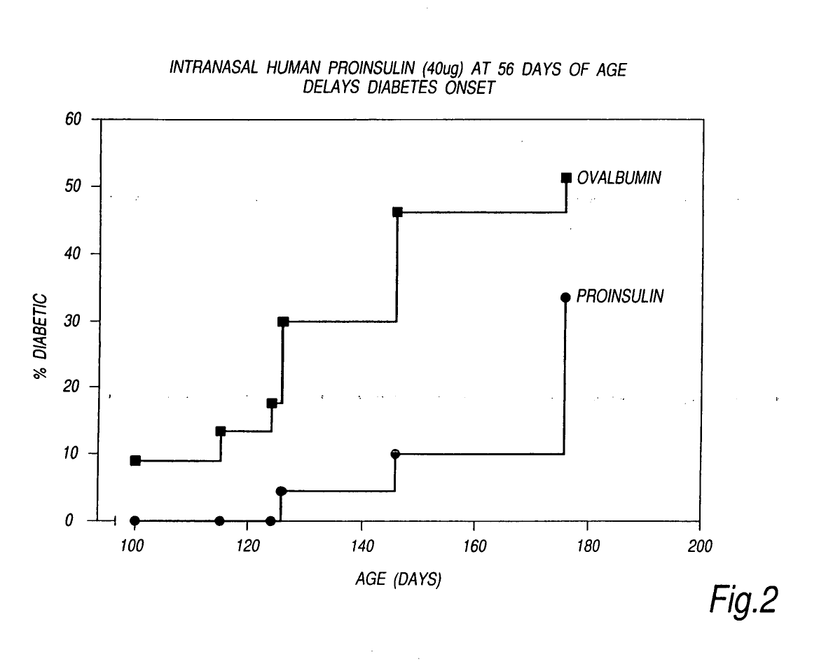

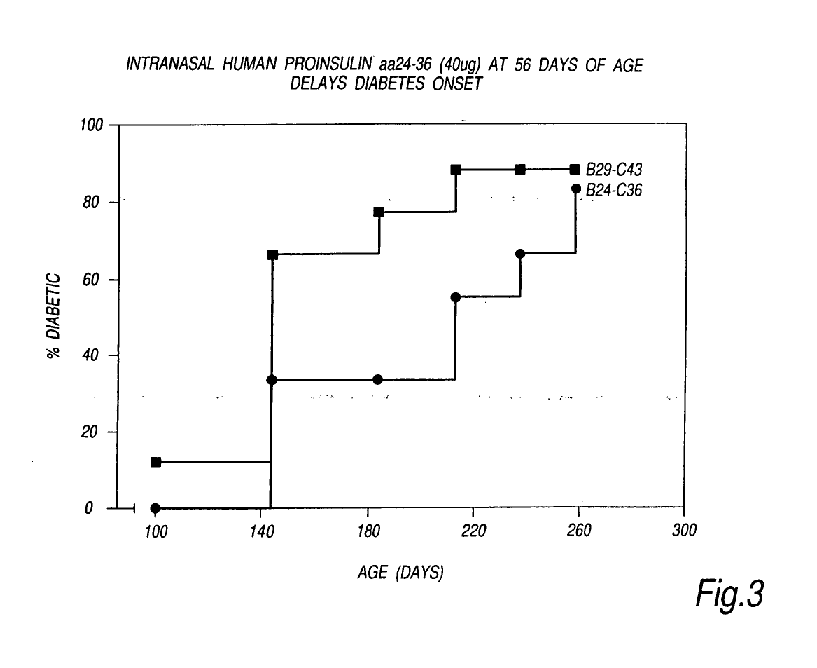

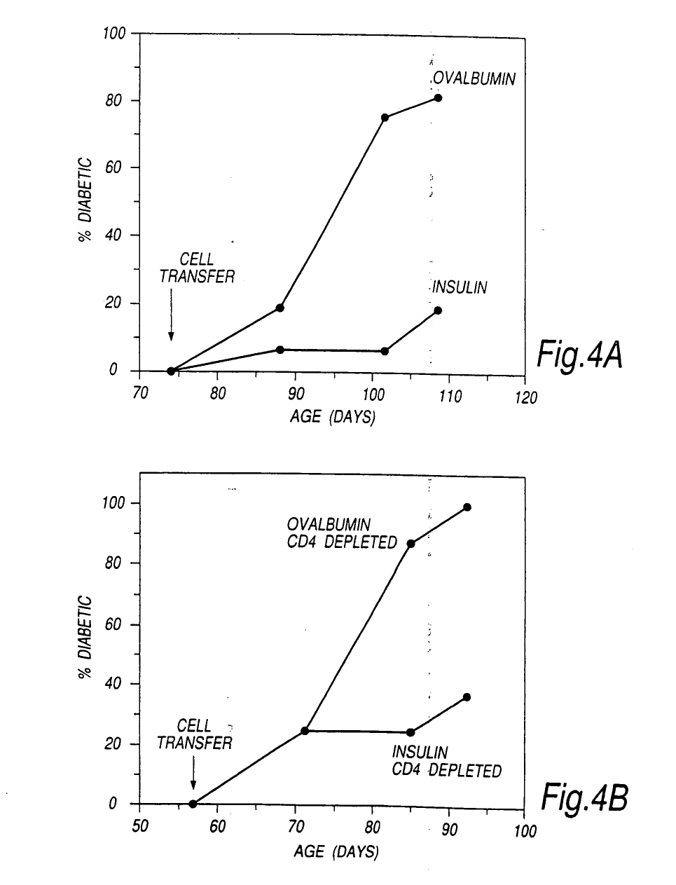

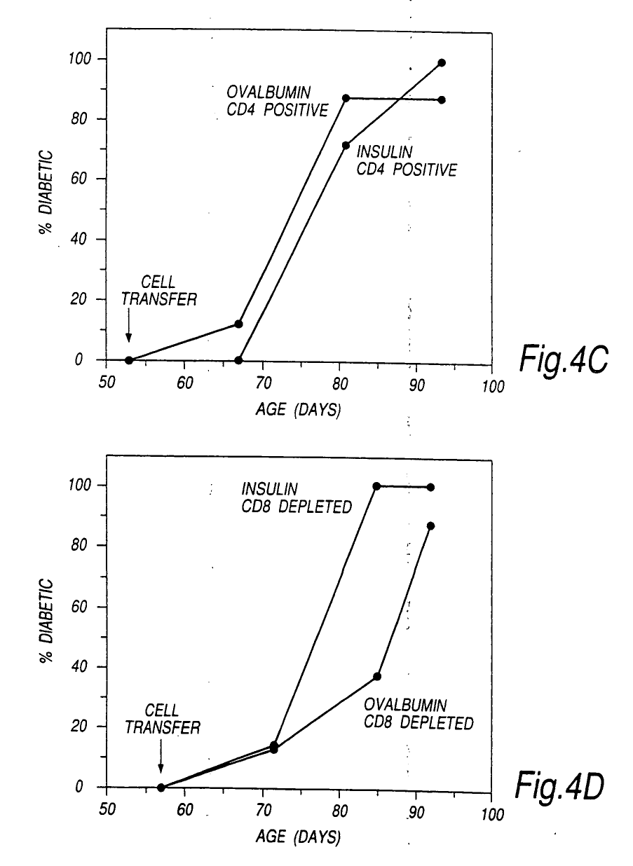

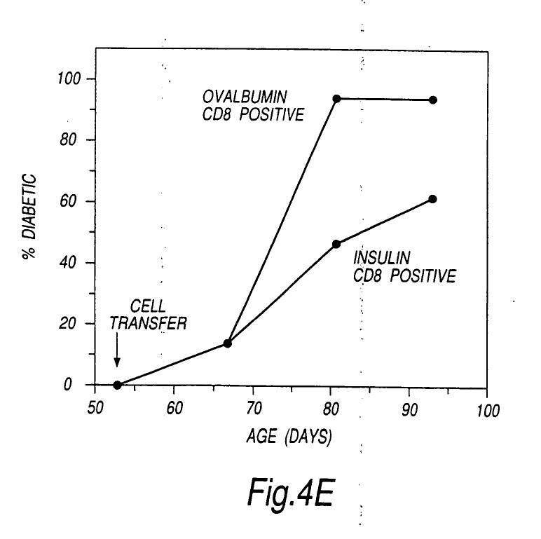

Spleen cells were then fractionated to identify the regulatory cells responsible for the suppression of diabetes transfer. Depletion and positive selection of CD4 and CD8 cells clearly showed that CD8 cells were wholly responsible for the suppression of transfer (FIG. 4B-3). Depletion of CD4 cells did not alter the ability of residual spleen cells from aerosol insulin mice to suppress transfer (FIG. 4B), and positively selected CD4 cells did not suppress transfer (FIG. 4C). On the other hand, there was no suppression by CD8-depleted spleen cells from aerosol insulin mice (FIG. 4D), whereas positively-selected CD8 cells suppressed transfer (FIG. 4E). The partial suppression by positively-selected CD8 cells, in contrast to the rapid development of diabetes after their depletion, is probably due to the inefficient recovery of CD8 cells; in this experiment, 7×10⁵ purified CD8 cells were co-injected into each recipient with 2×10⁷ spleen cells from diabetic mice.

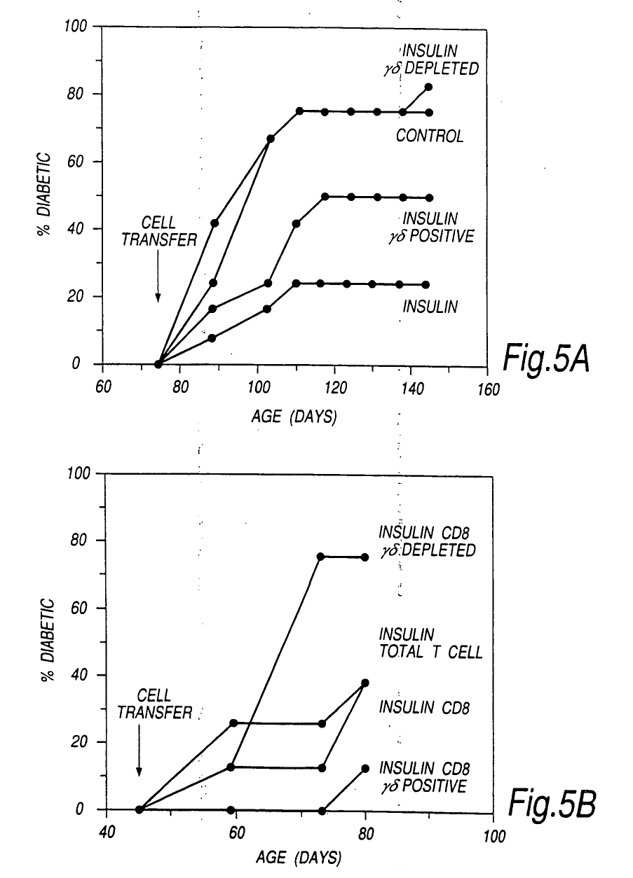

T cells bearing γδ receptors have been shown to hive an immunoregulatory role (14-19). Interestingly, it has been reported that total peripheral blood γδ cells decrease concomitantly with loss of β-cell function in humans with sub-clinical IDDM (20). To determine if the suppression of diabetes transfer that was observed was due to γδ T cells, the inventors fractionated spleen cells with the anti-γδ T-cell monoclonal antibody, GL3-1A (21). Depletion of γδ T cells, like that of CD8 cells, completely abrogated the ability of nylon wool non-adherent spleen cells from insulin aerosol-treated mice to suppress adoptive transfer of diabetes (FIG. 5A). Conversely, relatively small numbers of γδ T cells from insulin aerosol-treated mice could suppress transfer. Diabetes incidence after transfer was decreased by 50% for at least 70 days when 1.4×10⁵ γδ T cells were co-injected with 2×10⁷ spleen cells from diabetic mice (FIG. 5A). The spienic CD8 and γδ T cells that suppressed diabetes transfer were one and the same, and not two interdependent populations. Thus, the ability of CD8 cells from insulin aerosol-treated mice to suppress transfer was abolished if they were first depleted of γδ T cells, whereas small numbers of γδ cells purified from the CD8 cells prevented transfer (FIG. 5B). A summary of the results from 11 diflerent co-tratnsfer experiments is presented in FIG. 3.

FACS analysis revealed that γδ cells reactive with GL3 antibody constitute 1.6-2.4% of total and ˜1%, of CD8⁺ cells in the spleens of 12-16 week-old female NOD mice. These values were no different between groups of mice treated with insulin or ovalbumin aerosol. However, because of their low abundance distinct subpopulations of anitigen-specific CD8 γδ T cells would be difficult to distinguish this way. The higher protection with fractionated cells, for example sequentially-purified CD8 γδ cells (FIG. 6), is quantitative and reflects their higher absolute number relative to that in unfractionated cells.

### EXAMPLE 9

### Aerosolinization or Insulin

Aerosol inhalation as a mode of insulin delivery to the mucosa was as effective as oral insulin (22, 23) in reducing diabetes incidence in tile NOD mouse. The fact that it was therapeutic after the onset of insulitis is especially relevant to the prevention Of IDDM in at-risk humans with sub-clinical disease in whom the presence of circulating islet-antigen reactive antibodies and T cells is taken to reflect underlying insulitis. Indeed, compared to humans with recently-diagnosed IDDM, NOD mice have more intense insulitis and the majority of females to progress to diabetes (10, 11, 24). Aerosol insulin had no obvious metabolic effect but induced a population of regulatory CD8 γδ T cells, small numbers of which suppressed the ability of pathogenic effector T cells to adoptively transfer diabetes.

These antigen-induced ‘suppressor’ T cells protective against cell-mediated autoimmune pathology have not been previously described.

Oral tolerance has been associated with a decrease in cellular and sometimes an increase in humoral antigen-specific immunity, and with either CD8 or CD4 T cells that secrete, respectively, TGF-β or IL-4, IL-10 and TGF-β1 (8). However, these regulatory cells have not been identified as bearing γδ receptors. In NOD mice, oral tolerance to insulin was attributed to regulatory CD4 T cells (23). In accordance with the present invention CD8 γδ T cells account for the regulatory cells induced by aerosol insulin.

### EXAMPLE 10

### Intranasal Insulin, Proinsulin or Proinsulin Peptide 24-36

Intranasal insulin (FIG. 1), proinsulin (FIG. 2) or proinsulin peptide 24-36 (FIG. 3). Commercially available insulin at 4 mg/ml, or proinsuling or proinsulin peptide 24-36 at 1-4 mg/ml in either insulin carrier solution or mouse tonicity—phosphate buffered saline, was applied in a volume of 10-20 μl to the nostrils of unanaesthetised, restrained NOD female mice at either 28 or 56 days of age. Note that by 56 days of age all mice exhibit underlying islet inflammation (insulitis).

Single doses of insulin, proinsulin or 1proinsulin peptide 24-36 at either 28 or 56 days of age each significantly delayed the onset and decreased the incidence of diabetes in NOD female mice, compared to control proteins ovalbumin or hen egg lysozyme (HEL, not shown). On a comparative dose basis, proinsulin and proinsulin peptide 24-36 were more effective than insulin. These effects are greater with repeated doses of these proteins or peptide (not shown). In female mice pretreated with a single intranasal dose of proinsulin 24-36, whole splenocytes, and whole splenocytes depleted of CD8 but not CD4 T cells, significantly suppressed the adoptive transfer of diabetes by splenocytes from diabetic mice. Female mice were treated at 28 days of age, and then killed and their splenocytes taken from adoptive co-transfers at 56 days of age (FIG. 7).

### EXAMPLE 11

### Clinical Trial of Intranasal Insulin in At-Risk Individuals

A clinical trial involves administration of intranasal insulin to at-risk but otherwise healthy first-degree relatives with immune markers of IDDM, including circulating antibodies and T cells reactive with islet autoantigens. Our subjects have at least two antibodies, to insulin, GAD or tyrosine phosphatase IA-2, and peripheral blood T cell responses to insulin or proinsulin peptide 24-36, and sometimes to GAD and IA-2 peptides. The rationale is to induce mucosa-mediated immune tolerance to insulin, based on the success of this approach in the NOD mouse. Commercially-available human recombinant insulin is used, which is routinely given by subcutaneous or intravenous injection to people with IDDM. No significant side effects have been observed in 36 high-risk subjects aged 4-30 currently entered into the Trial. The possibility of mucosal irritation exists, but this has only been rare and then minor and transient. NOD mice treated with aerosol or intranasal insulin have exhibited no clinical complications, or abnormalities at autopsy.

This trial examines the effect of intranasal insulin on the surrogate immune markers of IDDM. The design is randomized, double-blind and placebo-controlled, with a crossover at six months. The placebo is the carrier solution normally used for insulin. The aim is to demonstrate significant effects on the levels of antibodies and T cells to insulin and other beta cell antigens. In addition, first phase insulin release in response to an intravenous injection of glucose, a measure of beta cell function, is monitored before, at six months and 12 months. The crossover design gives all subjects the opportunity of treatment (an important issue for at-risk relatives), measures if any treatment effects are sustained and allows within- and between-group analyses. Treatment is administered daily for 10 consecutive days, then for two consecutive days weekly. After six months, treatment is crossed over (from insulin to placebo, or vice versa).

The administration dose of insulin per nostril is approximately 200 μl of the commercial 4 mg/ml solution. The placebo is the carrier solution in which the insulin is normally dissolved.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Severity of insulitis and frequency of diabetes in NOD mice
treated with aerosol protein

| Protein | Insulitis score | Diabetes frequency |
|---|---|---|
| Insulin | 1.2 ± 0.98¹ | 2/32 (6.3%)² |
| Ovalbumin | 2.6 ± 0.92 | 8/32 (25%) |

Mice (32/group) were given either aerosol insulin or ovalbumin for 10 consecutive days and then weekly from 28 days of age. At 105 days of age, five non-diabetic mice from each group were killed for pancreas histology. The insulitis score is expressed as mean ±SD.
1. The insulitis score in insulin-treated mice with significantly reduced (p<0.01, Mann-Whitney U test)

2. The diabetes frequency in insulin-treated mice significantly reduced (p=0.04, Fisher's exact test)

TABLE 2
Proliferative responses of splenocytes from aerosol-treated mice

| | [³H]-thymidine uptake (cpm, mean ± SD) | |
|---|---|---|
| Additive | Insulin aerosol | Ovalbumin aerosol |
| None | 157 ± 28^{a} | 208 ± 29^{d} |
| Human insulin (40 μg/ml) | 134 ± 27 | 197 ± 82 |
| Mouse insulin II B-chain | 169 ± 80^{b} | 435 ± 240^{e} |
| (a 9-23) (40 μg/ml) | | |
| Human GAD65 (20 μg/ml) | 424 ± 165^{c} | 1381 ± 650^{f} |
| Concanavalin A (5 μg/ml) | 3357 ± 812 | 2960 ± 494 |
| Anti-CD3 antibody (10 μg/ml) | 2221 ± 533 | 2643 ± 1126 |

c ν a (p = 0.001), e ν d (p = 0.016), f ν d (p = 0.001), e ν b (p = 0.002), f ν c (p < 0.0001)

Splenocytes from three mice per group were assayed in quadruplicate in HL-1 serum-free medium. Statistical comparisons (Mann-Whitney U tests) were between the twelve results for each group.

TABLE 3
Cytokine secretion by splenocytes from aerosol-treated mice to
40 μg/ml of mouse insulin II B chain peptide (a 9-23)

| Cytokine | Insulin aerosol | Ovalbumin aerosol |
|---|---|---|
| IL-2 (pg/ml) | 200 ± 15 | 186 ± 27 |
| IFN-γ (pg/ml) | 368 ± 71 | 423 ± 31 |
| IL-4 (pg/ml) | 36 ± 6 | not detectable |
| IL-10 (pg/ml) | 222 ± 149 | not detectable |
| TGF-β | 293 ± 131 | 162 ± 41 |

Supernatants from replicate culture wells (Table 2) were sampled alter three days incubation and assayed for cytokines.

Bibliography:

1. Moses, A. C., et al. 1983. Diabetes 32:1040-1047.
2. Holt, P. G., et al. 1987. Immunol. Today 8:14-15.
3. Metzler, B., et al. 1993. Inter. Immunol. 5:1159-1165.
4. Waldo, F. B., et al. 1994. Clin. Immunol. Immunopathol. 73:30-34.
5. Kaufman, D. L., 1993. Nature 366:69-72.
6. Hurtenback, U., et al. 1989. J. Autoimmunity 2:151-161.
7. Hunt, P., et al. 1986. J. Immunol. 136:3994-3996.
8. Weiner, H. L., et al. 1994. Annu. Rev. Immunol. 12:809-837.
9. Tian, J., et al. 1996. J. Exp. Med. 183:1561-1567.
10. Honeyman, M. C. et al. 1993. Springer Semin. Immunol. 14:253-274.
11. Bach, J. F. 1994. Endocrine Rev. 15:516-542.
12. French, M. B., et al. 1997. Diabetes 46:34-39.
13. Wicker, L. S., et al. 1986. Diabetes (in press).
14. Kabelitz, D. 1992. Crit. Rev. Immunol. 11:281-303.
15. Kaufman, S. H. E., et al. 1993. Proc. Natl. Acad. Sci. USA 90:9620-9624.
16. Cardillo, F., et al. 1993. Eur. J. Immunol 23:2597-2605.
17. Gorczynski, R. M. 1994. Immunology 81:27-35.
18. Seo, N., et al 1995. Cancer Immunol. Immunother. 40:358-366.
19. . Nakata, R., et al. 1995. Immunol Immunopathol 3:217-222.
20. Lang, F. P., et al. 1993. J. Autoimmunity 6:107-119.
21. Goodman, T., et al. 1989. J. Exp. Med. 170:1569-1581.
22. Zhang, Z. H., et al. 1991. Proc. Natl. Acad. Sci. USA 88:10252-10256.
23. Bergerot, I., et al. 1994. J. Autoimmunity 7:655-663.
24. Kikutani, H., et al. 1992. Adv. Immunol. 51:285-322.
25. Adorini, L., et al. 1992. Springer Semin. Immunopathol. 14:187-199.
26. Muir, A. D., et al. 1993. Diabet. Metab. Rev,. 9:279-287.
27. Tisch, R., et al. 1994. Proc. Natl. Acad. Sci. USA 91:437-438.
28. Harrison, L. C. 1995. Mol. Med. 1:722-727.
29. Wells, H. G., 1911. J. Infect. Dis. 9:147-151.
30. Chen, Y., et al. 1994. Science 265:1237-1239.
31. Mordes, J. P., et al. 1987. Diab. Metab. Rev. 3:725-750.
32. Harrison, L. C. 1992. Immunol. Today. 13:348-352.
33. Dean, B. M., et al. 1986. Diabetologia 29:339-342.
34. Ziegler, A. G., et al. 1993. Diabetologia 36:402-408:
35. Harrison, L. C., et al. 1992. J. Clin. Invest. 89:1161-1165.
36. Rudy, G., et al. 1995. Mol. Med. 1:625-633.
37. Ziegler, A. G., et al. 1989. Diabetes 38:358-363.
38. Daniel, D., et al. 1986. Proc. Natl. Acad. Sci. USA 93:956-958.

1. A method of suppressing a cell-mediated autoimmune disease in a subject, said method comprising the administration as an aerosol of an effective amount of at least one antigen associated with said disease for a time and under conditions sufficient to prevent, reduce or otherwise ameliorate autoimmune pathology.

2. A method according to claim I wherein the autoimmune disease is insulin dependent diabetes mellitus (IDDM).

3. A method according to claim 1 wherein the autoimmune disease is slowly progressive IDDM (SPIDDM).

4. A method according to claim 1 wherein the autoimmune disease is gestational IDDM.

5. A method according to claim 1 wherein the antigen is selected from preproinsulin, proinsulin, proinsulin pC1)tide, insulin, glutamic acid decarhoxylase (GAD), tyrosine phosphatases IA-2, and derivatives or homologues thereof.

6. A method according to claim 5 wherein the proinsulin peptide is proinsulin peptide 24-36.

7. A method according to claim 5 wherein the amount of antigen administered is sufficient to induce or stimulate immunoregulatory mechanisms which are protective against cell mediated autoimmune pathology.

8. A method according to claim 7 wherein the immunoregulatory mechanism is production of suppressor T cells.

9. A method according to claim 8 wherein the antigen is a proinsulin peptide and suppressor T cells are CD4 T cells.

10. A method according to claim 9 wherein the suppressor T cells are CD4αβ T cells.

11. A method according to claim 7 wherein the proinsulin peptide is proinsulin peptide 24-36.

12. A method according to claim 8 wherein the antigen is insulin and the suppressor T cells are CD8 T cells.

13. A method according to claim 12 wherein the suppressor T cells are CD8γδ T cells.

14. A method according to claim 5 wherein more than one antigen is administrated simultaneously or sequentially.

15. A method according to claim 14 wherein a proinsulin peptide is administered simultaneously or sequentially with one or more of insulin, GAD, tyrosine phosphatase IA-2, preproinsulin or proinsulin.

16. A method according to claim 14 wherein proinsulin or a derivative or homologue thereof is administered simultaneously or sequentially with a disease suppressing peptide comprising the proinsulin 24-36 sequence

| | |
|---|---|
| FFYTPKTRREAED. | [SEQ ID NO:1] |

17. A method for the prophylaxis or treatment of IDDM, SP1DDM or gestational IDDM comprising the administration as an aerosol of one or more of proinsulin, proinsulin peptide, preproinsulin, insulin, GAD or tyrosine phosphatase IA-2, or derivative or homologue thereof for a time and under conditions sufficient to prevent, reduce or otherwise ameliorate autoimmune pathology of IDDM, SPIDDM or gestational IDDM.

18. A method according to claim 17 wherein the proinsulin peptide is proinsulin peptide 24-36.

19. A method according to claim 17 wherein a proinsulin peptide is administered simultaneously or sequentially with one or more of insulin, GAD, tyrosine phosphatase IA-2, proinsulin or preproinsulin.

20. A method according to claim 17 wherein proinsulin or a derivative or homologue thereof is simultaneously or sequentially administered with a disease suppressing peptide comprising the proinsulin 24-36 sequence

| | |
|---|---|
| FFYTPKTRREAED. | [SEQ ID NO:1] |

21. A method according to claim 1 wherein the antigen or combination of antigens are delivered by aerosol form at a rate of from 1 to about 20 litres/min.

22. A method according to claim 20 wherein the amount of antigen administration is from about 0.1 μg to about 5 mg.

23. A composition comprising proinsulin or a derivative or homologue thereof in a form suitable for aerosol administration and one or more pharmaceutically acceptable carriers and/or diluents.

24. A composition according to claim 23 further comprising insulin.

25. A composition according to claim 23 further comprising proinsulin peptide 24-36.

26. A composition according to claim 23 further comprising a disease suppressing peptide comprising the proinsulin 24-36 sequence

| | |
|---|---|
| FFYTPKTRREAED. | [SEQ ID NO:1] |

27. An agent for the prophylaxis and treatment of IDDM, SPIDDM or gestational IDDM wherein said agent induces or stimulates immunoregulatory mechanisms, said agent comprising proinsulin or a derivative or homologue thereof in a form suitable for aerosol administration and one or more pharmaceutically acceptable carriers and/or diluents.

28. An agent according to claim 27 wherein said immunoregulatory mechanism is production of suppressor T cells.

29. An agent according to claim 27 further comprising one or more of insulin, proinsulin peptide 24-36 and a disease suppressing peptide comprising the proinsulin 24-36 sequence

| | |
|---|---|
| FFYTPKTRREAED. | [SEQ ID NO:1] |

30. Use of proinsulin or a derivative or homologue thereof in the manufacture of a medicament for the prophylaxis or treatment of IDDM, SPIDDM or gestational IDDM.

31. Use according to claim 30 further comprising the use of one or more insulin, proinsulin peptide 24-36 and a disease suppressing peptide comprising the proinsulin 24-36 sequence

| | |
|---|---|
| FFYTPKTRREAED. | [SEQ ID NO:1] |
